(19) United States
(12) Patent Application Publication
(10) Pub. No.: US 2007/0160743 A1
(43) Pub. Date: Jul. 12, 2007

(54) Method for coating biocompatible material on a substrate

(76) Inventors: John L. Babitt, Minnetonka, MN (US); Alex Alden Peterson, Maple Grove, MN (US); Jacquie M. Campbell, Minneapolis, MN (US)

Correspondence Address: Haugen Law Firm PLLP, 1130 TCF Tower, 121 South Eighth Street, Minneapolis, MN, 55038, US

(21) Appl. No.: 11/327,950

(22) Filed: Jan. 9, 2006

(51) Int. Cl. A61L 33/00; B05D 3/00; B05D 7/00; C08F 2/46

(52) U.S. Cl. 427/2.1; 427/421.1; 427/487

(57) ABSTRACT

A method for coating a material on targeted surfaces, such as on an implantable medical device, includes providing a spray application apparatus that defines an application zone within which a spray device is disposed, and supplying the spray device with a material solution including an activatable species. The object having the surfaces targeted for coating is placed within the application zone and subjected to the concurrent application of the material solution and a stimulus, with the stimulus operably activating the activatable species.

### FIELD OF THE INVENTION

The present invention relates to medical device coatings generally, and more particularly to a method for applying biocompatible material coatings to implantable medical devices.

### BACKGROUND OF THE INVENTION

A large number of surgical procedures now involve the implantation of artificial medical devices into the patient's body. The implantation of such medical devices, however, introduces risks of complications related to having a foreign body surgically affixed within the patient. Such complications include, for example, thrombogenic action caused by the foreign body response of the patient which can lead to fibrous deposition on device surfaces.

In an effort to minimize the likelihood of such complications, coatings have been developed for application to surfaces of the respective medical devices exposed to the body of the human patient when positioned in vivo. Typically, such coatings have focused on masking the device to render it more biocompatible to resist or suppress the thrombogenetic response of the patient. Example biocompatible materials include heparin, albumin, and streptokinase.

A variety of coating procedures have been employed to coat surfaces of implantable medical devices with biocompatible materials. Typical coating techniques include brush, dip, ultrasound, dauber, micro-fine spraying, vacuum deposition, and the like. Conventional coating techniques, however, are most effective in coating standard open and freely accessible surfaces of the medical devices. When the coating application involves restricted access, tight tolerances, and cavities, known coating techniques have the tendency to result in non-uniform thicknesses and qualities. Moreover, conventional coating methods may also exhibit inconsistent coating results from device to device.

Accordingly, it is a principal object of the present invention to provide a coating technique for coating biocompatible materials on implantable medical devices, which technique results in uniform and reproducible coating qualities.

It is a further object of the present invention to provide a method for coating biocompatible materials on implantable medical devices that is effective in obtaining desired coating quality in restricted access locations of the medical device.

It is another object of the present invention to provide a method for applying a coating to an implantable medical device, which method reduces the amount of time required to create a uniform coating.

### SUMMARY OF THE INVENTION

By means of the present invention, a method is provided for obtaining a surface coating that may be applied in uniform and reproducible fashion, and particularly throughout complex surfaces of implantable medical devices. The method of the present invention preferably facilitates the application of biocompatible materials as a uniform coating layer on implantable medical devices. Such a coating procedure involves the concurrent application of a biocompatible material solution having activatable species incorporated therewith, and a stimulus that activates the activatable species.

In a particular embodiment, the method for coating a material on an implantable medical device includes providing a spray application apparatus that defines an application zone within which a spray device is disposed, and supplying the spray device with a material solution including an activatable species. An implantable medical device is placed within the application zone such that the material solution and a stimulus are concurrently applied to the implantable medical device, with the stimulus operably activating the activatable species.

In another embodiment, the method enables the coating of a solution-based material on targeted surfaces of an implantable medical device by providing a spray apparatus and supplying the spray apparatus with the material solution and a photo-activatable linking agent. The implantable medical device is placed within an application zone of the spray apparatus, and the material solution and photo-activatable linking agent are applied in spray form to the targeted surfaces while simultaneously exposing the application zone to ultraviolet radiation.

### BRIEF DESCRIPTION OF THE DRAWINGS

FIG. 1 is a schematic view of a spray application apparatus of the present invention; and

FIG. 2 is a flow diagram of a coating method of the present invention.

### DETAILED DESCRIPTION OF THE PREFERRED EMBODIMENTS

The objects and advantages enumerated above together with other objects, features, and advances represented by the present invention will now be presented in terms of detailed embodiments described with reference to the attached drawing figures which are intended to be representative of various possible configurations of the invention. Other embodiments and aspects of the invention are recognized as being within the grasp of those having ordinary skill in the art.

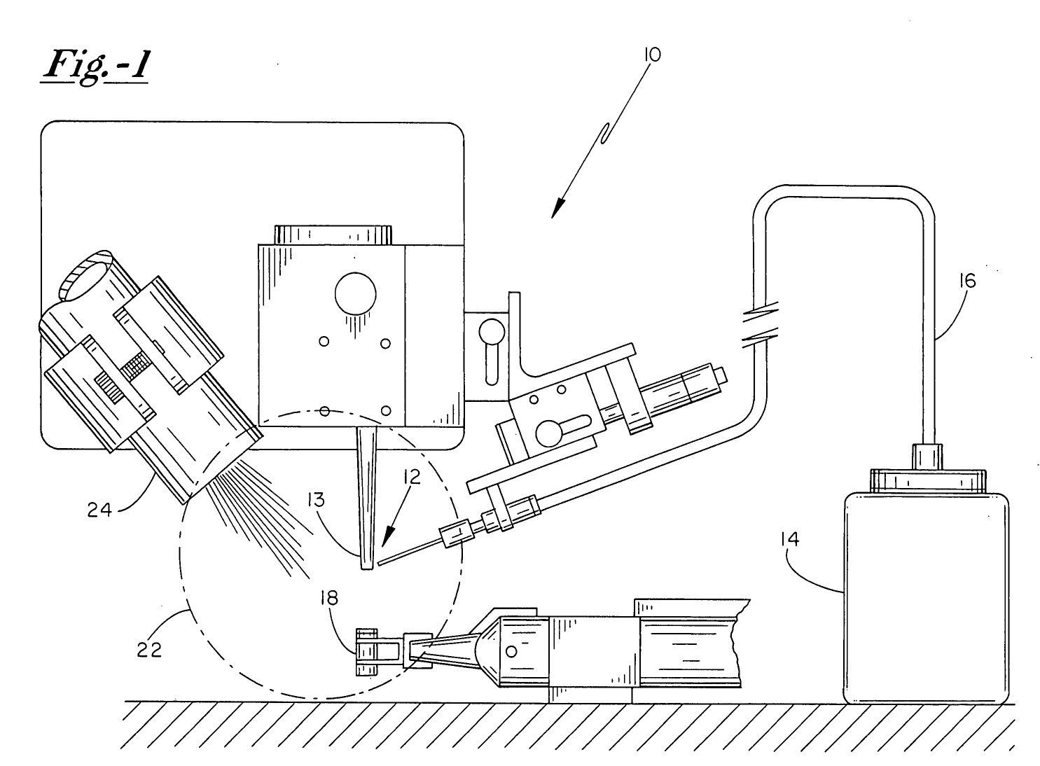

With reference now to the drawing figures, and first to FIG. 1, a spray application apparatus 10 is configured for operably coating one or more materials on devices such as implantable medical devices. Apparatus 10 preferably includes a spray device 12 that is fluidly coupled to a liquid supply device 14 for operably supplying spray device 12 with material to be deposited upon medical device 18. An example spray device 12 useful in apparatus 10 of the present invention is available from Sono-Tek Corporation of Milton, N.Y. under the trade name “MicroMist”.

Liquid supply device 14 preferably includes a fluid reservoir and a fluid pump for pumping liquid from the reservoir into supply line 16 at a desired fluid flow rate. In preferred embodiments, liquid supply device 14 may be a syringe pump available from KD Scientific, Inc. of Holliston, Mass. utilizing a 2.5 syringe from Hamilton Medical Products, Inc. of Mill Valley, Calif. Other types of liquid supply devices, however, are contemplated as being useful in the spray application apparatus of the present invention.

Supply line 16 is preferably configured for conveying fluids therethrough, and is preferably fabricated of a durable and inert material, such as PTFE or the like. A wide variety of materials and sizes for supply line 16 are contemplated as being useful in supplying spray device 12 with liquid from liquid supply device 14.

Spray device 12 is preferably operably positioned within an application zone, identified in FIG. 1 by dashed area 22. Application zone 22 is defined by apparatus 10, and is particularly defined by the space between an outlet section 13 of spray device 12 and medical device 18, as well as outlet section 13 of spray device 12 and medical device 18. Application zone 22 may be partially or fully enclosed, but is preferably open, as is illustrated in FIG. 1. Outlet section 13 of spray device 12, therefore, may be fixed in place, or may be manually or automatically positionably adjustable to desired locations within application zone 22.

The MicroMist system preferably provides for a spray droplet size range of about 38 μm at a delivery pressure of about 13 psi. The MicroMist system preferably utilized in apparatus 10 of the present invention incorporates an ultrasonic nozzle on which surface the coating liquid is applied so as to generate the desired droplet size. A gas stream operating at about 13 psi flows through the ultrasonic nozzle so as to transport liquid droplets from the ultrasonic nozzle tip to medical device 18 in spray form. In preferred embodiments, the ultrasonic generator for creating ultrasonic vibrations at the nozzle operates at a frequency of 48 kHz, and is driven by 1 W of power. One of ordinary skill in the art, however, would readily understand that substitute or additional devices capable of emitting liquid in spray form may be incorporated into apparatus 10 of the present invention.

Preferably, spray application apparatus 10 further includes a means for providing a stimulus to application zone 22. Various forms of stimulus such as thermal energy, radiant energy, chemical reaction, and the like are contemplated as being useful in the method of the present invention. In a particularly preferred embodiment, apparatus 10 includes an ultraviolet radiation-emitting lamp 24 that is positioned to supply ultraviolet radiation to application zone 22. In a particular embodiment, ultraviolet radiation emitting lamp 24 may be in the form of a Bluewave™ 200 ultraviolet lamp available from Dymax Corporation of Torrington, Conn. Preferably, ultraviolet radiation emitting lamp 24 is capable of producing an intensity of at least about 80 mW/cm² within a wavelength range of between about 320 and 390 nm at medical device 18 disposed within application zone 22.

Thermal energy may also be a preferred mode of stimulus provided to application zone 22. It has been found by the Applicants that a temperature of at least about 50° C. may assist in the coating procedure of the present invention. Accordingly, the stimulus applied to application zone 22 may be in the form of thermal energy alone, or combinations of thermal energy, radiant energy, and/or other forms of stimulus. In a particularly preferred embodiment of the present invention, a combination of ultraviolet radiation and thermal energy is directed to application zone 22 of apparatus 10 preferably during both the coating stage and the cure stage of the process.

In preferred embodiments, spray device 12 sprayably applies an activatable material upon medical device 18 in the presence of a stimulus that activates such activatable material, and thereby cures such material as a coating layer upon medical device 18. In an exemplary embodiment, the activatable material being deposited on medical device 18 is a biocompatible material to render medical device 18 biocompatible when implanted in vivo. A variety of biocompatible materials are useful in application upon implantable medical devices, with heparin representing a commonly utilized anti-thrombogenic biocompatible material.

Many biocompatible materials, including anti-thrombogenic factors, are applicable to medical devices in solution form. Traditionally, activatable species such as photo-activatable compounds have been incorporated into the biocompatible material-containing solutions, such that the solution may be cured to fix the biocompatible material in place on the medical device. It has surprisingly been found by the Applicants, however, that the biocompatible materials may be effectively and efficiently fixed in a uniform and consistent layer upon the medical device through the sprayable application of such biocompatible material-containing solutions in the presence of an appropriate stimulus. Applicants theorize that the method of the invention partially cures the solution prior to contact with the medical device, and therefore rapidly accelerates the cure time of the solution when disposed upon the medical device to obtain a consistent and reproducible coating quality. As such, the stimulus is preferably most directly focused at the solution spray stream and the targeted surfaces of the medical device during the coating procedure.

Although a variety of arrangements of the component parts within application zone 22 may successfully obtain desired coating qualities, a preferred arrangement positions the outlet section 13 of spray device 12 between about 0.25 and 5 cm from the targeted surfaces of medical device 18, and more preferably between about 1 and 2 cm therefrom. Additionally, the stimulus emitting mechanism, such as ultraviolet radiation lamp 24, is spaced from the solution spray stream and medical device 18 by between about 0.5 and 5 cm, with the radiation output from lamp 24 being directed at the solution spray stream and medical device 18.

As indicated above, the coating solution for use in the present invention preferably incorporates a biocompatible material. In a particular embodiment, such a biocompatible material may be an anti-thrombogenic factor such as heparin, albumin, streptokinase, urokinase, tissue plasminogen activator (TPA), and the like. Moreover, the coating solutions of the present invention preferably include at least one activatable agent for assisting in covalently bonding the anti-thrombogenic factor to the medical device. In some embodiments, a parylene tie-layer coating is disposed upon respective surfaces of the medical device, such that the anti-thrombogenic factor coating is covalently bonded to the parylene tie-layer coating and/or directly upon the surfaces of the medical device. Examples of cross-linking agents and tie-layer coatings useful in the present invention are described in U.S. Pat. Nos. 5,002,582; 5,512,329; 6,077,698; 6,278,018; 6,603,040; and 6,706,408, the contents of which are herein incorporated by reference. Example formulations for the biocompatible material solution are described in, for example, U.S. Pat. Nos. 4,973,493 and 4,979,959, the contents of which are herein incorporated by reference. A vast array of formulations other than those described in the above-cited patents are contemplated as being useful in coatings created by the methodology of the present invention.

Medical device 18 may be disposed in a stationary or movable orientation within application zone 22. Various devices may be employed to move medical device 18 with respect to spray device 12, with such devices including, for example, rotatable spindles, conveyors, movable shelves, and the like. It is understood by the Applicants that those of ordinary skill in the art may customize apparatus 10 as illustrated in FIG. 1 within the known boundaries of the conventional art.

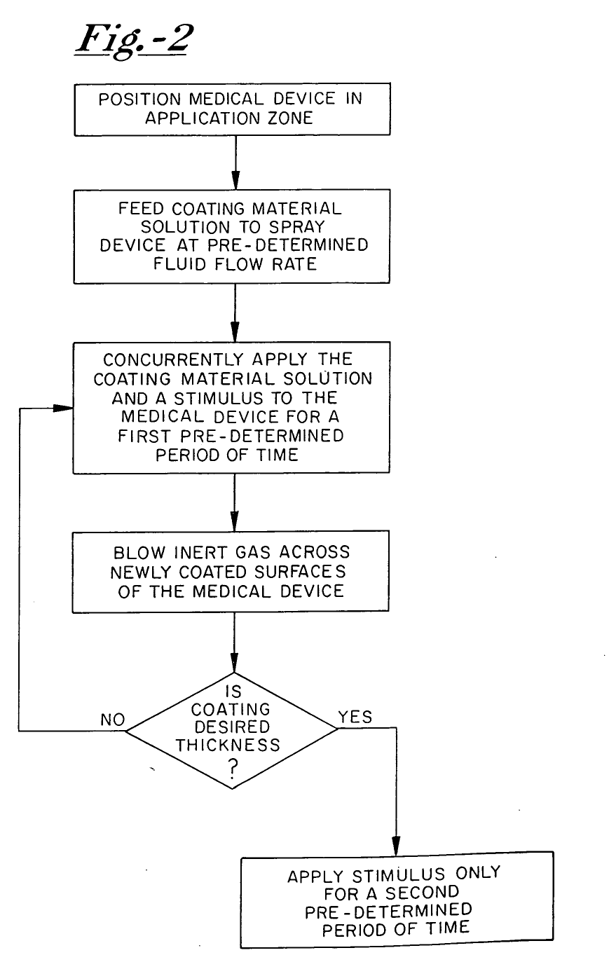

As illustrated in the flow diagram of FIG. 2, a medical device is prepared for the coating operation of the present invention by placing the device in application zone 22 of apparatus 10. Spray device 12 is supplied with a coating material solution at a desired flow rate, such that the coating material solution is emitted from the spray device at a predetermined volumetric flow rate. The coating material solution and a stimulus are then concurrently applied to the medical device within the application zone for a predetermined period of time sufficient to coat the targeted surface of the medical device to a desired degree.

An inert gas such as nitrogen may be blown across the newly coated surfaces of the medical device in order to remove any excess debris, and to further smooth the coatings. In some embodiments, this coating procedure is repeated as many times as necessary to obtain a desired final coating thickness. Once the coating has achieved a desired thickness on medical device 18, a stimulus is again applied to medical device 18 for a second predetermined period of time without application of the coating material solution in order to fully activate the activatable species within the coating material solution, and to thereby fully cure the coating upon the targeted surface of the medical device.

Without limiting the scope of the invention, the following example is provided to demonstrate a preferred mode of the method of the present invention.

### EXAMPLE

A coating material solution was prepared incorporating polyvinylpyrrolidone (PVP) available from Surmodics, Inc. of Eden Prairie, Minn. under the chemical identifier PV05 at a concentration of 6 mg/ml; a photo-activatable cross-linking agent available from Surmodics under the chemical identifier PR04 at a concentration of 0.05 mg/ml; and modified heparin having photo-activatable groups molecularly bound thereto and available from Surmodics under the chemical identifier HP01 at a concentration of 2 mg/ml; with the mixture being solvated in 100% de-ionized water.

A MicroMist spray application apparatus from Sono-Tek was located in a clean room environment having a temperature of 20° C. and a relative humidity of 40%. A Dymax Bluewave™ 200 ultraviolet lamp was utilized and calibrated to provide an intensity value of 80 mW/cm². The ultrasonic spray head on the spray device was calibrated for a gain of 1.0 watts, and was supplied with Nitrogen gas at 13 psi for output at the spray head nozzle.

The prepared solution was placed in a 2.5 ml Hamilton syringe, and pumped through a supply line with a KD Scientific #210 pump having a volumetric output flow rate set at 0.05 ml/min.

A titanium vessel connector tube segment having a conventional parylene tie layer coating and an outside diameter of 6 mm was placed in the spray application apparatus at a position 1.5 cm from the outlet section of the spray head, and in alignment with the fluid output of the spray head and the radiation emission of the ultraviolet lamp. The solution was emitted from the spray head concurrently with the illumination of the ultraviolet lamp for 37.5 seconds. The spray application was ceased at the expiration of 37.5 seconds, and a flow of nitrogen gas was then blown across the coated surface at a pressure of 13.0 lbs/in² for 5 seconds. This process was repeated several times to achieve a desired coating thickness.

The ultraviolet lamp was energized to illuminate the coated tube segment for 50 seconds. The coating was then measured to be of adequate uniformity and heparin activity. The coated tube segment was evaluated through pressure pulsation testing and combined pressure/displacement testing, with SEM photographs taken before and after the testing showing that the coating was well adhered to the tube segment. In addition, an ethylene oxide sterilization regimen was undertaken with the heparin activity of the coating remaining stable.

The invention has been described herein in considerable detail in order to comply with the patent statutes, and to provide those skilled in the art with the information needed to apply the novel principles and to construct and use embodiments of the invention as required. However, it is to be understood that the invention can be carried out by specifically different devices and that various modifications can be accomplished without departing from the scope of the invention itself.

What is claimed is:

1. A method for coating a solution-based material on targeted surfaces of an implantable medical device, the method comprising:
(a) providing a spray apparatus;
(b) supplying said spray apparatus with said material solution and a photo-activatable linking agent;
(c) placing said implantable medical device in an application zone; and
(d) applying said material solution and said photo-activatable linking agent in spray form to said targeted surfaces while simultaneously exposing said application zone to ultraviolet radiation.

2. A method as in claim 1 wherein said material is an anti-thrombogenic factor.

3. A method as in claim 1 wherein said material includes a photo-activatable group chemically linked thereto.

4. A method as in claim 1 wherein an outlet section of said spray apparatus is spaced from said implantable medical device in said application zone by between about 0.25 and 5 cm.

5. A method as in claim 1, including applying said material solution to said targeted surfaces at a volumetric rate of between about 0.01 and 0.1 ml/min and an outlet gas pressure from said spray apparatus of between about 10 and 15 lbs/in².

6. A method as in claim 1 wherein the ultraviolet radiation has an intensity of at least about 80 mW/cm² in said application zone.

7. A method as in claim 1 wherein said targeted surfaces include a chemical bonding layer.

8. A method for coating a material on an implantable medical device, the method comprising:
(a) providing a spray application apparatus defining an application zone within which a spray device is disposed;
(b) supplying said spray device with a material solution including an activatable species;
(c) placing said implantable medical device in said application zone; and
(d) concurrently applying said material solution and a stimulus to said implantable medical device, with said stimulus operably activating said activatable species.

9. A method as in claim 8 wherein said activatable species is photo-activatable.

10. A method as in claim 8 wherein said activatable species is chemically linked to said material.

11. A method as in claim 8 wherein said stimulus is ultraviolet radiation.

12. A method as in claim 11 wherein the ultraviolet radiation has an intensity of at least about 80 mW/cm² in said application zone.

13. A method as in claim 8 wherein said material is biocompatible.

14. A method as in claim 13 wherein said biocompatible material is an anti-thrombogenic factor.

15. A method as in claim 8 wherein said spray device is spaced from said implantable medical device in said application zone by between about 0.25 and 5 cm.

16. A method as in claim 8, including laterally moving said spray device with respect to said implantable medical device during application of said biocompatible material solution, with the relative lateral motion having a rate of about 100 mm/s.

17. A method as in claim 8, including passing an inert gas through said application zone at predetermined intervals.

18. A method as in claim 8, including applying said stimulus for a predetermined period of time following application of said biocompatible material solution to said implantable medical device.

19. A method as in claim 8 wherein said stimulus is thermal energy.

20. A method as in claim 8 wherein said stimulus includes ultraviolet radiation and thermal energy.

21. A method as in claim 19 wherein said application zone is maintained at a temperature of at least about 50° C.

22. A method as in claim 20 wherein said application zone is maintained at a temperature of at least about 50° C.
